(19) United States
(12) Patent Application Publication
(10) Pub. No.: US 2025/0099114 A1
(43) Pub. Date: Mar. 27, 2025

(54) Eccentrically rotating cutting tool

(71) Applicant: Michael Smith, Excelsior, MN (US)

(72) Inventor: Michael Smith, Excelsior, MN (US)

(21) Appl. No.: 18/896,866

(22) Filed: Sep. 25, 2024

Related U.S. Application Data
(60) Provisional application No. 63/540,813, filed on Sep. 27, 2023.

(51) Int. Cl. A61B 17/17; A61B 17/00; A61B 17/16

(52) U.S. Cl. CPC A61B 17/17; A61B 17/1613; A61B 2017/00473

(57) ABSTRACT

A surgical tool for and method of removing a bone wherein a working end of the tool eccentrically rotates with one or more cutting edges, such that in a first position the one or more cutting edges is shielded by one or more surfaces of the distal end of the main body of the tool and in a second position at least one or more of the one or more cutting edges are exposed for cutting a bone in contact therewith. The main body has a foot plate which guides positioning of the working end and protects soft tissue and adjacent structures while removing bone.

### CROSS-REFERENCE TO RELATED APPLICATION

The present application claims priority to and the benefit of U.S. provisional application Ser. No. 63/540,813, filed on Sep. 27, 2023, the contents of which are hereby incorporated in their entirety.

### BACKGROUND

The present disclosure relates generally to a handheld surgical device configured for performing one or more surgical procedure(s) and more specifically to a device configured to cut away small pieces of bone during one or more surgical procedure(s).

A Kerrison punch is a commonly used tool in spinal surgery and has been around for over 100 years. Using the punch requires repetitive squeezing of the handle to nibble away bone during the course of surgery. The punching action of the Kerrison punch can pinch and tear unintended tissue including nerves, blood vessels, and/or the spinal cord. Additionally, the repetitive squeezing action is fatiguing to the surgeon and consequently, the chance of pinching or tearing tissue increases as fatigue and hand cramping occur.

Using the punch to nibble away bone is a slow process with a typical sequence of inserting the footer under the edge of the bone to be removed, squeezing the handle to pressure punch a nubbin of bone, removing the tool from the site, wiping the removed bone out of the tool, and rinsing and repeating this sequence, sometimes hundreds of times, until enough bone is removed to consider that part of the procedure complete.

### SUMMARY

An aspect of the present disclosure relates to a surgical tool for cutting bone comprising a main body comprising a proximate end and a distal end, with a length extending therebetween wherein the distal end has an opening therein and terminates in a foot plate; a rotatable shaft operably supported within the main body; a working end provided on a distal end of the rotatable shaft and positioned at least partially with the opening in the distal end of the main body, wherein the working end comprises one or more cutting edges, and wherein the working end eccentrically rotates such that in a first position the one or more cutting surfaces of the working end are protected by one or more surfaces of the distal end of the main body and in a second position one or more of the one or more surfaces of the distal end are exposed.

The foot plate is a vertical foot plate positioned at an angle with respect to a horizontal axis of the length extending between the proximate and distal ends of the main body.

In one or more embodiments, the foot plate is provided at an angle in the range of about 30 degrees to about 90 degrees or more.

In one or more embodiments the working end comprises a plurality of cutting edges spaced apart on a length of the working end. The working end comprises a plurality of cutting edges at one or more locations on the length of the working end.

The distal end of the surgical tool has a rounded or arcuate shape and the opening spans a top and at least one side of the distal end.

Another aspect of the present disclosure relates to a method of removing bone. Removing the bone includes inserting a distal end of a surgical device into a selected space between a portion of bone to be removed and adjacent tissue and using an angled foot plate of the distal end of the surgical device to guide the surgical device into the selected space and/or protect the adjacent tissue when removing bone. Actuating the surgical device and rotating a working end of the surgical device cyclically exposes one or more cutting surfaces to the bone for cutting the bone and sliding the surgical device along the bone allows for continuing to remove bone without removing the surgical device from the selected space or an adjacent selected space.

When inserting the distal end of the surgical device into the space, the cutting surfaces of the working end are present in the distal end of the surgical device but remain shielded by one or more surfaces of the distal end.

Yet another aspect of the present disclosure relates to a surgical tool with a working end comprising an opening and one or more shielding surfaces for one or more cutting surfaces eccentrically rotatable about a longitudinal axis of the working end such that when the one or more cutting surfaces are stationary, the one or more cutting surfaces are shielded by the one or more shielding surfaces and when the one or more cutting surfaces are rotating, the one or more cutting surfaces are cyclically exposed at the opening, the working end further comprising a foot plate for guiding insertion of the surgical tool, protecting one or more structures adjacent an area to be contacted with the one or more cutting surfaces, or a combination thereof.

The foot plate is positioned at an angle with respect to the longitudinal axis of the working end, the angle being in the range of about 30 to about 90 degrees.

In one or more embodiments, the foot plate is removable from the working end. In one or more embodiments the foot plate is integral with and otherwise not removable from the working end.

The surgical tool comprises a main body housing a rotatable shaft for operable connection to the one or more cutting surfaces of the working end.

In one or more embodiments, the working end is removable from the main body.

In one or more embodiments, the working end is integral to the main body and/or rotatable shaft.

### BRIEF DESCRIPTION OF THE DRAWINGS

FIG. 1 illustrates a powered rotary cutting tool.

FIG. 2 is a cross sectional side view of the cutting tool with a working end in a parked position.

FIG. 3 is a perspective view of the cutting tool with the working end in a running position.

FIG. 4 is a top perspective view of the working end.

FIG. 5 is a top perspective view of another embodiment of the working end.

FIG. 6 is a top perspective view of yet another embodiment of the working end.

FIG. 7 is a bottom perspective view thereof.

FIG. 8 is a side perspective view of another embodiment of the working end.

FIG. 9 illustrates positioning of the cutting tool between a bone spur and adjacent structures.

### DETAILED DESCRIPTION

This disclosure includes a surgical tool. The surgical tool is a powered rotary tool providing an axially eccentric cutting edge or edges. As described herein the powered rotatory tool is designed for the removal of bone. One or more cutting edges of the tool are rotatable to remove a portion of bone during a surgical procedure. While the device of this disclosure is exemplified in connection with the removal of bone, the device and the concepts associated therewith may be used for and/or applied to any surgical procedure requiring cutting and/or removal of tissue or other structures from a patient.

The device includes a main body that allows the device to be hand held. The main body of the device generally has a first, proximal end for gripping and a second, distal end having an opening for access to a working end of a rotatable shaft. The distal end further terminates in a footer plate. The footer plate may be positioned at a terminal end of the main body, providing an end wall to the opening which provides access to or otherwise exposes the working end.

The footer plate may be angled with respect to the main body, allowing the device to be slid into a space between a bone and an adjacent structure. The footer plate may further act as a guide for positioning the bone for contact with the exposed cutting edge or edges. Further, the device may be slid along a bone to remove selected areas of bone, eliminating the need for removal and/or cleaning of the device between cuts.

The main body houses the rotatable shaft, and an end of the shaft comprises the working end. The working end as described herein comprises one or more cutting edges also referred to as cutting surfaces. The working end of the shaft rotates within the distal end of device, but as the working end rotates, the cutting edges of the working end are exposed at the opening or openings in the distal end of the main body of the device for cutting bone. The opening in the distal end may be provided in an upper surface and/or along one or more sides of the distal end.

In further detail, the working end may be an axially eccentric working end. When the device is powered off, the working end is positioned precisely such that the cutting edge or edges are provided in a set, parked position. In this parked position, the working end is substantially encased or otherwise shielded within the distal end of the main body and the cutting edge(s) are not exposed. The distal end may be tubular in that the working end is accommodated therein to at least some extent. The distal end may be rounded or arcuate on at least one exterior surface. For example, the distal end may have a cross-sectional shape that is arcuate along at least a portion, for example a bottom portion. This allows the device to be positioned within a wound or surgical site and/or otherwise slid into a location proximate the bone for cutting without damage to adjacent structures. When actuated, the cutting edge or edges rotate and during rotation the edge or edges are cyclically exposed at and/or extend through the opening. This allows exposure of the cutting edge(s) and direct contact between the cutting edge(s) and the bone being cut when the working end is rotated. The working end does not retract into a length of the main body to shield the working end.

In one or more embodiments the distal end of the main body comprises a thin foot plate, the foot plate having a reduced thickness when compared to hand punch tools. The foot plate is thin enough to be slid into position with minimal disturbance to adjacent structures, yet thick enough to house the cutting edge and protect adjacent structures from being damaged. The footplate has a thickness that may vary and/or be proportional to a diameter of the cutting edge(s) and/or of the working end. The thickness may vary with respect to the varying diameter or size of the cutting edge(s) and/or vary for fitting into spaces while withstanding manual force applied for positioning and/or cutting. The distal end may terminate in this upright footplate, with or without side walls along the distal end. The foot plate may further be angled with respect to an axis along a length of the main body of the device.

The foot plate may have a thickness in the range of about 0.025 inches (about 0.65 mm) to about 0.075 inches (about 2.1 mm) and/or is otherwise to sufficient to reduce the chance of inadvertently catching soft tissue or compressing underlying structures during use of the device. In another embodiment the foot plate has a thickness of less than approximately 0.075 inches. The thickness may also vary in order to withstand force applied when fitting the distal end in a space near the bone to be cut and/or in combination with the size of the cutting surface(s). The distal end of the device does not comprise a hemispheric bowl nor does a sleeve of the device terminate in such a bowl. The device instead comprises the distal end which terminates in the angled foot plate and which may have an upper opening and one or more side openings. In one or more embodiments, the distal end comprises a floor and the foot plate. The floor may have an arcuate length. In some embodiments, the distal end comprises the floor, foot plate and one or two opposing side walls. The foot plate may be angled with respect to an axis extending along a length of the device such that the foot plate may be positioned at approximately 45-degree angulation to the long axis of the sleeve. However, the foot plate can be constructed with varying degrees of angulation from 0 to 90 degrees with respect to the long axis, including but not limited to 90 degrees or 30 degrees.

The foot plate may further be integral to or removably coupled to the distal end of the device. For example, the foot plate may be removable in that the device can be provided as a system having multiple foot plates where the foot plates may have the same or different construction or dimensions. Thus, if a foot plate is damaged it can easily be replaced. Additionally, or alternatively, foot plates of different thickness and/or angle can be interchanged for tailored cutting and/or to match patient anatomy. Mechanisms for removably securing the foot plate to the distal end include but are not limited to frictional engagements, sliding channels having mating dimensions, snaps, screws or the like.

The handle portion of the device comprises a motor operably connected to the shaft having the working end. The motor is powered to rotate the shaft and the working end such than an offset opening exposes the rotating cutting surface to bone and removes it. The motor may be powered electrically, for example AC/DC powered, pneumatically powered, or vacuum driven. The motor may be housed within the handle or otherwise operably connected to the shaft housed therein.

In one or more embodiments, the device may be a battery powered device wherein the hand-held device comprises a motor, along with control software for actuating the motor. The battery may also be provided within the hand-held piece or otherwise operably couplable to the hand-held piece. Where the hand-held device is battery powered and the working end for the device may be attachable to the rotary shaft for use. The working end may then, in one or more embodiments, have interchangeable cutting surface(s).

The device is generally configured to accommodate typical rotation speeds of the shaft and thus the working end which may vary, but may generally be in the range of about 60,000-75,000 RPM range. The device is further configured to “park” the working end in a selected docked or parked position which does not expose the cutting edge(s) outside of the main body of the device to allow the foot plate to be positioned as needed, for example, under the bone or other structure to be cut. Once actuated and the working end is rotating or spinning, the cutting edge(s) then are exposed at or through opening(s) in the distal end and thus rotate into contact with the bone to rake or cut the bone.

In further detail, the working end of the device may comprise one, two, three or more cutting edges or surfaces. Generally, the working end and/or the cutting edges is/are constructed from medical grade materials such as stainless steel. The cutting edge(s) may be configured to eccentrically rotate.

The working end may include a small amount of metal ground away opposite the cutting end or grind end to allow for shaft balancing.

A backstop may also be provided on the working end to provide rigidity to a location on the foot plate, allowing the foot plate to have a decreased thickness.

In use, the device may be used for side cutting a bone or a burr for example. Additionally, the bone may be fed into the top and/or side of the distal end for contact with the rotating working end for removal of bone. The footer plate may aid in this sliding placement. The device may be inserted under the stock, such as a bone spur, and positioned between a bone spur and the adjacent structure such as a pinched nerve there below. As the working end is rotated, the spur is cut away and the nerve there below is protected by at least a portion of the distal end of the device.

The bone debris from the cutting action, also referred to as “swarf” is similar in appearance to sawdust and when using the device described herein can be suctioned away from the bone. In contrast to prior art devices where the device must be removed and cleaned for each cut, the device described herein allows the working end to be gently slid to a subsequent area for bone removal without removal of the tool from the wound or surgical site. The constant in and out passes with the prior art cutting tool are eliminated and the procedure time greatly reduced. The device of this disclosure is configured to generally increase the safety of the procedure.

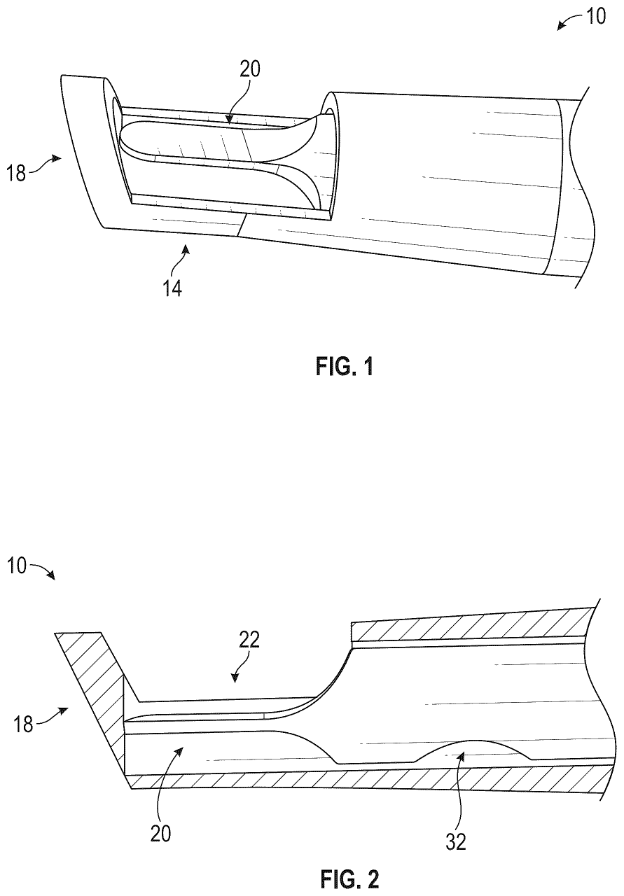

Illustrated in FIGS. 1-4 is a handheld surgical device 10 and working end 20 thereof. The handheld surgical device 10 has a main body 11 with a proximal end 12 and a distal end 14. The proximal end 12 includes a handle 16 configured for an operator's hand to hold, preferably comfortably, while the handheld surgical device 10 is being used. The distal end 14 terminates in a foot plate 18 and includes a working end 20. At least a part of the working end 20 is configured for being inserted into a wound or an anatomical area to perform a function such as the cutting of bone during a surgical procedure.

The proximal end 12 of the handheld surgical device 10 supports a switch disposed at or near the handle 16. Although not shown, the switch may be disposed at a different position, such as along the length of the device 10 or at the distal portion 14. The switch is connected to a control unit in the handle 16 connected to a motor, which in turn is connected to a rotating shaft 30 inside a length or neck portion of the device 10. The working end 20 may include a small amount of metal ground away 32 opposite the cutting end or grind end to allow for shaft balancing.

When the switch is activated, a control unit powers the motor for rotating the shaft 30. As the shaft terminates in or is otherwise coupled to the working end 20, the working end 20 rotates with the rotatable shaft 30.

The distal end 14 comprises, and may terminate with a foot plate 18, which is angled with respect to the axis 19 extending along the length of the main body 11. One or more openings 22 in the distal end 14 expose the working end 20 of the device 10 and as the working end 20 rotates, exposes the one or more cutting edges 24 of the working end 20. The working end 20 comprises one, two, three or more cutting edges 24.

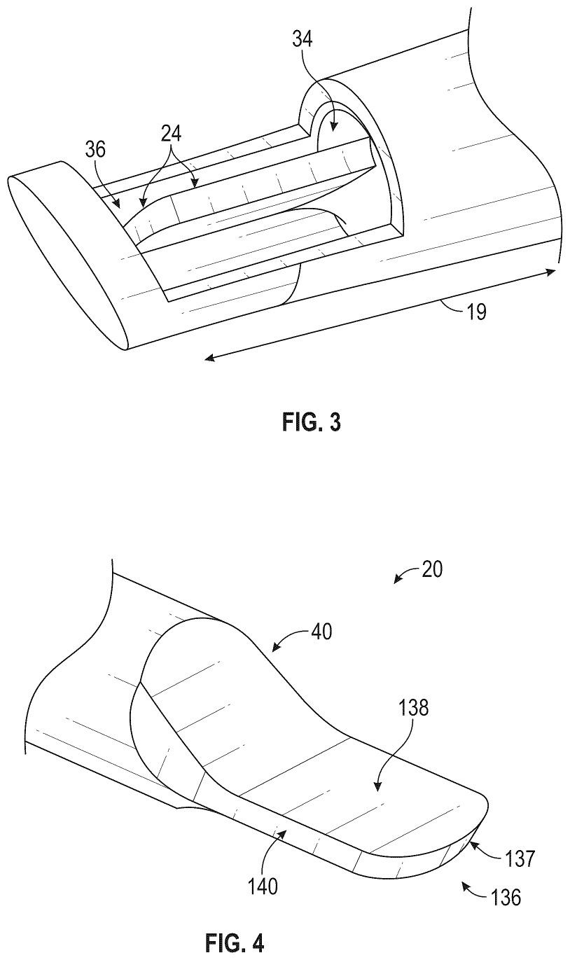

In a first, parked position as illustrated in FIGS. 1 and 2 the cutting edge(s) 24 is/are contained within or otherwise not exposed by or through the opening 22. The cutting edge(s) 24 are protected by sides of the distal end 14 from contacting the bone or other internal structures by sides 24 of the distal end 14 and the foot plate 18. In a second, driving position as illustrated in FIG. 3 the working end 20 rotates and thus exposes the one or more cutting edges 24 at or through the opening 22 cyclically as the working end 20 is rotated. The cutting edge(s) 24 are rotated to be exposed at or along the opening 22 in the distal end 14 of the device 10, and as the cutting edge(s) is/are exposed and rotated, bone or structures in contact therewith is cut away.

In further detail, the working portion 20 is located at or near a distal end of the rotating shaft 30 or is otherwise connected to a distal end of the shaft 30. In some embodiments, the working portion 20 is removably attached to the shaft so that the working portion 20 may be replaced.

The working end 20 has a proximal end 34 opposite its own distal end 36. The working end 20 may have one, two, three or more cutting edge(s) or surfaces 24 and these cutting edges or surfaces 24 may be positioned at or near the distal end 36 of the working end 20 and/or along a length 38 of the working end 20. The length 38 of the working end 20 may taper away from the proximal end 34 of the working end 20 and the rotating shaft 30 at a shoulder 40. This allows the cutting edge(s) or surfaces 24 to rotate eccentrically as the length 38 has a cross-section that at one or more locations is smaller than the cross section of the shoulder 40 and rotating shaft 30. A cross-section of the length 38 may or may not be symmetrical.

As illustrated in further detail in FIG. 4, the working end 20 may have a substantially flat length 138 where the distal end 136 thereof is rounded for providing a grinding surface 24 which acts as a cutting end when rotated. Similarly, side edges 140 of the flat length 138 are exposed when the working end 20 is rotated for cutting. In FIGS. 1 and 2, this working end is in a parked, unexposed position and in FIG. 3, the cutting edge is rotated to expose the cutting edge(s) 24. As shown in further detail in FIG. 4, the distal end 136 may terminate in a tapered edge 137 to provide a cutting edge 24 at the protruding or forward edge 137.

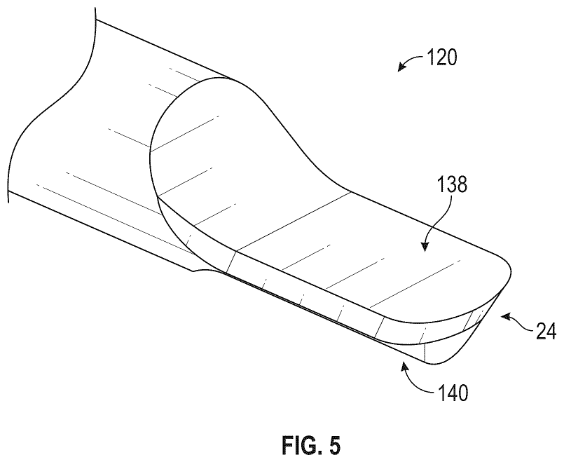

As illustrated in further detail in FIG. 5, in one or more embodiments the working end 120 may further comprise a lip 140 which extends outwardly along one side of the length 138. This channel 140 has a width smaller than a width of the length 138 and may be positioned to one side or in the center of the length 138 and co-extend partially or substantially along the length 138. This channel 140 also is exposed as a cutting edge 24 when the working end 120 is rotated.

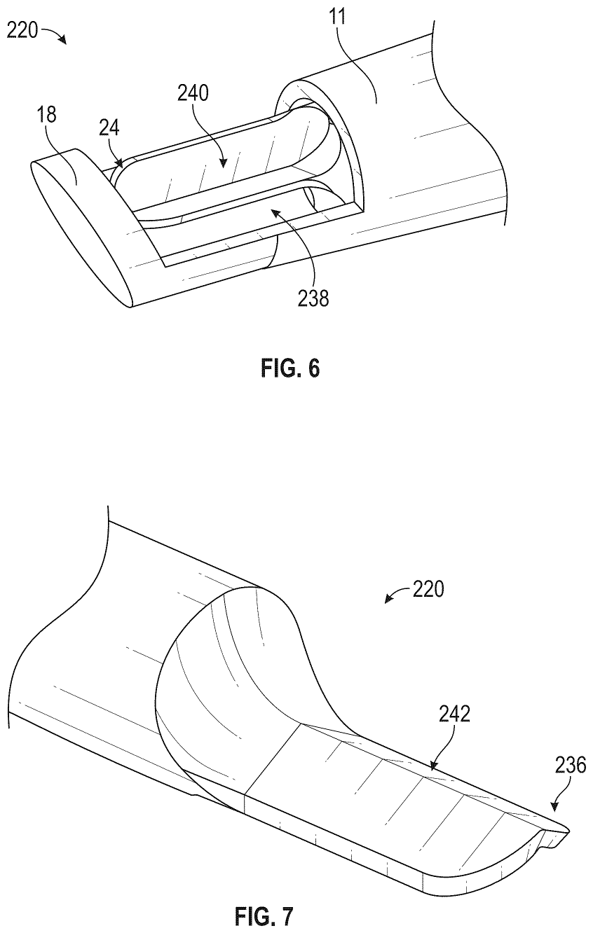

Another embodiment of the working end is illustrated at 220 in FIGS. 6 and 7. The working end 220 may alternatively have a concave length 238 with a rounded area at the proximal end of the working end 20. Along the length 238 the concave section may be smooth and rounded or as illustrated have angled sloping sides to provide a “v” shaped channel 240 along the length 238 such that the cutting edges or surfaces 24 are provided on the perimeter sides and bottom apex 242 of the length 238 that are exposed as the working end 220 is rotated.

The channel 240 comprises a pitched surface providing cutting edges 24 formed by abutting sloping surfaces with an angle of 10 degrees, 20 degrees or more. The working end 220 may be considered bi-planar with a central ridge at the highest point. The forward edge 236 of the working end 220 may be rounded.

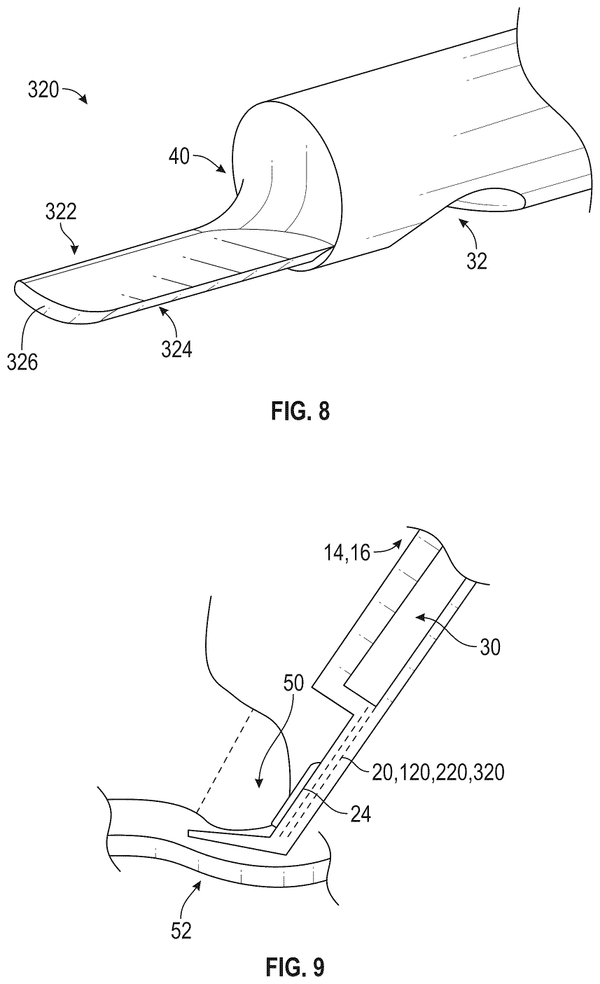

Another embodiment of the working end is illustrated at 320 in FIG. 8. The working end 320 may comprise a rounded rear edge 322 and angled front edge 324 which provides a cutting surface 24 when rotated in the distal end 14 of the main body of the device 10. A forward edge 326 may be tapered and rounded to further provide another cutting edge 24 when the working end 320 is rotated. A length 338 of the working end 320 may be off-center in positioning as length 338 extends from the rotating shaft 30 such that when the working end 320 is in the parked position in the device 10, the distal end 14 has one or more surfaces which obscure the cutting edges 24 and when the working end 320 is rotated, the edges 24 are exposed for cutting matter in contact therewith as illustrated in FIG. 9.

FIG. 9 illustrates one method of positioning the device 10 with the working end 20, 120, 220, 320 between material to be cut, such as a bone spur 50 and adjacent structures such as nerves 52. The distal end 14 is slid along the bone spur 50 to remove the excess bone as the working end 20, 120, 220, 320 is rotated and exposes the cutting edge(s) 24 to remove the bone in contact therewith.

Although the present disclosure has been described with reference to preferred embodiments, workers skilled in the art will recognize that changes may be made in form and detail without departing from the spirit and scope of the disclosure.

1. A surgical device for cutting bone comprising:
a main body comprising a proximate end and a distal end, with a length extending therebetween wherein the distal end has an opening therein and terminates in a foot plate;
a rotatable shaft operably supported within the main body;
a working end provided on a distal end of the rotatable shaft and positioned at least partially with the opening in the distal end of the main body, wherein the working end comprises one or more cutting edges, and
wherein the working end eccentrically rotates such that in a first position the one or more cutting surfaces of the working end are protected by one or more surfaces of the distal end of the main body and in a second position one or more of the one or more surfaces of the distal end are exposed.

2. The surgical device of claim 1 wherein the foot plate is a vertical foot plate positioned at an angle with respect to a horizontal axis of the length extending between the proximate and distal ends of the main body.

3. The surgical device of claim 2 wherein the foot plate is provided at an angle in the range of about to 30 degrees to about 90 degrees or more.

4. The surgical device of claim 1 wherein the foot plate has a thickness of less than approximately 0.075 inches.

5. The surgical device of claim 1 wherein the working end comprises a plurality of cutting edges spaced apart on a length of the working end.

6. The surgical device of claim 1 wherein the working end comprises a plurality of cutting edges at one or more locations on the length of the working end.

7. The surgical device of claim 1 wherein the distal end of the main body has an arcuate shape and the opening spans a top and at least one side of the distal end.

8. The surgical device of claim 1 wherein the foot plate is separable from the main body.

9. A method of removing bone comprising:
inserting a distal end of a surgical device into a selected space between a portion of bone to be removed and adjacent tissue;
using an angled foot plate of the distal end of the surgical device to guide the surgical device into the selected space and/or protect the adjacent tissue when removing bone;
actuating the surgical device and rotating a working end of the surgical device to cyclically expose one or more cutting surfaces to the bone for cutting the bone; and
sliding the surgical device along the bone to continue removing bone without removing the surgical device from the selected space or an adjacent selected space.

10. The method of claim 9 wherein when inserting the distal end of the surgical device into the space the cutting surfaces of the working end are present in the distal end of the surgical device but remain shielded by one or more surfaces of the distal end.

11. A surgical tool with a working end comprising an opening and one or more shielding surfaces for one or more cutting surfaces eccentrically rotatable about a longitudinal axis of the working end such that when the one or more cutting surfaces are stationary, the one or more cutting surfaces are shielded by the one or more shielding surfaces and when the one or more cutting surfaces are rotating, the one or more cutting surfaces are cyclically exposed at the opening, the working end further comprising a foot plate for guiding insertion of the surgical tool, protecting one or more structures adjacent an area to be contacted with the one or more cutting surfaces, or a combination thereof.

12. The surgical tool of claim 11 wherein the foot plate is positioned at an angle with respect to the longitudinal axis of the working end, the angle being in the range of about 30 to about 90 degrees.

13. The surgical tool of claim 11 wherein the foot plate is removable from the working end.

14. The surgical tool of claim 11 and further comprising a main body housing a rotatable shaft for operable connection to the one or more cutting surfaces of the working end.

15. The surgical tool of claim 14 wherein the working end is removable from the main body.
